(19) United States
(12) Patent Application Publication
(10) Pub. No.: US 2025/0288770 A1
(43) Pub. Date: Sep. 18, 2025

(54) MEDICAL VENTILATOR DISPLAYING THE AIRWAY OPENING PRESSURE OF THE PATIENT

(71) Applicant: Air Liquide Medical Systems, Antony (FR)

(72) Inventors: Arnaud LESIMPLE, Antony (FR); Laura POLARD, Antony (FR); Mathilde LEFRANC, Antony (FR); Jean-Christophe RICHARD, Sevrier (FR); Eloise DE BEAUFORT, Bagneux (FR)

(21) Appl. No.: 19/080,446

(22) Filed: Mar. 14, 2025

(30) Foreign Application Priority Data
Mar. 15, 2024 (FR) FR2402621

(51) Int. Cl. A61M 16/20; A61M 16/00

(52) U.S. Cl. CPC A61M 16/202; A61M 16/0066; A61M 2205/3334; A61M 2205/3337; A61M 2205/3355; A61M 2205/505; A61M 2230/46

(57) ABSTRACT

The invention relates to a medical ventilator (1) comprising gas supply means (2) for supplying a respiratory gas to a patient circuit (3), pressure measurement means (4), flowrate determination means (5), data processing means (10) for processing the pressure and/or flowrate measurements, display means (11), and storage means (10.3) for storing a pressure-volume compliance curve (C1) of the circuit, representing the compliance of the respiratory circuit. The data processing means (10) are configured to perform a determination of the AOP, and the display means (11) display the AOP value (20). The AOP is used to adjust a PEP valve (13) arranged on the patient circuit (3).

### CROSS REFERENCE TO RELATED APPLICATIONS

This application claims the benefit of priority under 35 U.S.C. § 119 (a) and (b) to French Patent Application No. 2402621, filed Mar. 15, 2024, the entire contents of which are incorporated herein by reference.

### BACKGROUND

The invention relates to a medical ventilator, that is to say a respiratory assistance apparatus capable of supplying a respiratory gas, such as air, oxygen or an air/oxygen mixture, making it possible to determine and display the opening pressure of the patient's airways or the AOP (airway opening pressure) of a patient receiving the respiratory gas.

Acute respiratory distress syndrome (ARDS) is a common cause of death in humans. Its management requires the application of what is called “protective” ventilation of the patients, which is generally implemented by a respiratory assistance apparatus, also called a medical ventilator, with customization of the ventilation parameters for each patient, typically the flowrate, the pressure and/or the frequency of the respiratory cycles.

A phenomenon known as airway closure has been reported in 23 to 52% of patients suffering from acute respiratory distress syndrome (ARDS).

Therefore, detection of airway closure and measurement of the airway opening pressure are essential for adapting the ventilation to each patient.

In fact, setting a positive expiratory pressure (PEP) lower than the airway opening pressure may detrimentally affect the measurement of the respiratory mechanics of the patient.

In addition, cyclic opening and closing of the small airways may also occur and promote development of ventilator-induced lung damage.

There is therefore a risk to patients when the ventilation parameters are chosen incorrectly or inadequately.

Other medical ventilators are described in WO2020/080986, U.S. Pat. No. 5,738,090, FR3137296, US2011/023881, US2019/275276 and US2015/100917.

A problem is therefore to be able to determine a possible closure of the airways in patients, in particular ARDS patients, and to measure the airway opening pressure (AOP) when this closure of the airways is present, in order to avoid misinterpretation of the curves and calculations of respiratory mechanics and also to customize the ventilation based on the analysis of the phenomenon of airway closure, so as to avoid or minimize the risks to the patients.

### SUMMARY

A solution of the invention is a medical ventilator, i.e. a respiratory/ventilatory assistance apparatus, for supplying a respiratory gas to a patient, comprising:
gas supply means for supplying a respiratory gas,
a patient circuit supplied with respiratory gas by the gas supply means,
pressure measurement means configured to determine the pressure of the gas in the patient circuit,
flowrate determination means configured to determine the flowrate of respiratory gas in the patient circuit,
data processing means comprising at least one (micro) processor for processing the pressure and/or flowrate measurements performed by the pressure measurement means and the flowrate determination means,
display means controlled by the data processing means, and
storage means for storing a pressure-volume compliance curve of the circuit (P-Vcircuit_patient), representing the compliance of the respiratory circuit.

In addition, the medical ventilator further comprises selection means, which can be actuated by the user in order to start a determination of the airway opening pressure (AOP), and the data processing means are configured to perform a determination of the AOP in response to an actuation of the selection means by the user:
a) by controlling the gas supply means to supply the respiratory gas at a “low” respiratory rate of between 2 and 10 ventilation cycles per minute, and at a “slow” insufflation flowrate of between 2 and 10 l/min, until an inspiratory volume of between 200 and 1500 ml or a maximum pressure (Pmax) of between 20 and 60 cmH₂O is obtained,
b) by controlling a display, on the display means, of the stored pressure-volume compliance curve of the circuit (P-Vcircuit_patient) and a pressure-volume curve corresponding to the “slow” flowrate insufflation (P-Vflowrate_slow), the stored “slow” flowrate insufflation pressure-volume curve (P-Vcircuit_patient) being displayed superimposed on the pressure-volume compliance curve of the circuit (P-Vcircuit_patient),
c) by searching for and locating, on the “slow” flowrate insufflation pressure-volume curve (P-Vflowrate_slow), an inflection point corresponding to a change in slope of said “slow” flowrate insufflation pressure-volume curve (P-Vflowrate_slow) with separation of the “slow” flowrate insufflation pressure-volume curve (P-Vflowrate_slow) from the pressure-volume compliance curve of the circuit (P-Vcircuit_patient), and
d) by determining the pressure value at the inflection point located on the “slow” flowrate curve (P-Vflowrate_slow).

In addition, the data processing means are further configured to control a display, on the display means, of the AOP value, i.e. the opening pressure of the patient's airways, corresponding to the pressure value determined at the present inflection point of the “slow” flowrate curve (P-Vflowrate_slow).

In the context of the invention, in order to simplify its understanding, the following abbreviations and/or simplifications are used:
“AOP” to designate the airway opening pressure of the patient;
“compliance curve” to designate the pressure-volume compliance curve of the circuit (P-Vcircuit_patient);
“slow flowrate curve” to designate the “slow” flowrate pressure-volume curve (P-Vflowrate_slow).
“% vol.” means percentage by volume.
“O₂” designates oxygen.
the terms “means of/to/for” are considered to be wholly equivalent to and capable of being substituted by the terms “device of/to/for”, for example the term “data processing means” may be replaced by “data processing device”, the term “display means” may be replaced by “display device”, the term “storage means” may be replaced by “storage device”, etc.

Depending on the embodiment in question, the medical ventilator of the invention can comprise one or more of the following features:
the patient circuit comprises an inspiratory branch and an expiratory branch.
the gas supply means supply the respiratory gas at the insufflation flowrate for a duration sufficient to obtain the desired inspiratory volume of between 200 and 1500 ml or the desired maximum pressure (Pmax) of between 20 and 60 cmH₂O.
said sufficient duration derives from, i.e. depends on or is a function of, the setting of the desired flowrate and the desired inspiratory volume.
said sufficient duration is preferably less than 60 seconds, for example typically between 1 and 45 seconds.
the inspiratory branch and the expiratory branch are fluidically connected at a joining piece, typically a Y-shaped piece.
the patient circuit is fluidically connected to a respiratory interface, such as a breathing mask, an endotracheal intubation tube or other.
the inspiratory branch supplies the respiratory gas to the respiratory interface.
the respiratory gas contains oxygen (O₂) in a proportion of at least 20 vol. %, preferably at least 21 vol. %. approximately.
the respiratory gas is air, oxygen or an air/oxygen mixture.
the inspiratory branch is fluidically connected to the gas supply means, in particular to the gas supply outlet of a motorized turbine.
the expiratory branch recovers the gas exhaled by the patient in the respiratory interface, that is to say the gas containing CO₂.
the respiratory gas supply means comprise a respiratory gas source, such as a motorized turbine.
alternatively, the respiratory gas supply means comprise means for fluidic connection to a respiratory gas source, such as a wall-mounted gas outlet.
the data processing means comprise at least one (micro) processor for determining one or more gas volumes entering or leaving the ventilator on the basis of the processing performed on the flowrate measurements performed by the flowrate determination means.
the pressure measurement means comprise a pressure sensor.
the flowrate determination means comprise a flowrate sensor.
the pressure measurement means are arranged in the inspiratory branch of the patient circuit.
the pressure measurement means are arranged inside the ventilator, i.e. in its casing.
the display means are configured to show displays, in particular one or more curves, numerical values, texts, messages, windows, icons, etc., or any other information.
the data processing means are further configured to control a display, on the display means, of information relating to the existence of a closure of the patient's airways, i.e. in particular when an inflection point is detected and an AOP is determined, for example a text message indicating the presence/existence or absence of an airway closure, for example “presence of airway closure” or “airway closure detected” or, conversely, “absence of airway closure” or “airways open”, or any other equivalent message.
the data processing means are further configured to control a display, on the display means, of a text message indicating the pressure value “x” corresponding to the determined AOP, that is to say in the case of the presence of airway closure, for example “AOP=x cmH₂O”, with x typically between 1 and 30 cmH₂O.
data processing means comprising at least one (micro) processor implementing one or more algorithms.
the data processing means comprise a microcontroller.
said at least one (micro) processor is arranged on an electronic board.
the data processing means are configured to determine the inhaled volume and the exhaled volume.
the display means comprise a touch screen, that is to say a touch panel.
the selection means comprise a virtual key displayed on the display means, in particular on the touch screen, that is to say a touch key, i.e. a key actuated by pressing it with a finger.
the touch screen is a colour display.
the “low” respiratory rate is between 3 and 7 ventilation cycles per minute, for example of the order of 5 ventilation cycles per minute.
the “slow” insufflation flowrate is between 3 and 7 l/min, for example of the order of 5 l/min.
the data processing means are configured to control a display of the AOP value in the form of a positive pressure value, preferably expressed in cmH₂O or in another unit.
the slow flowrate curve is obtained from the pressure and flowrate values processed by the microprocessor(s).
the “slow” flowrate curve is a curve displayed and updated in real time.
the compliance (e.g. the stored compliance curve) is obtained before the start of treatment of the patient.
the compliance of the patient circuit is stored before the start of treatment of the patient, during tests to verify the correct functioning of the machine or “self tests”. This compliance of the circuit is then used to determine and display the compliance curve (i.e. P-Vcircuit_patient curve) superimposed on the slow flowrate curve (i.e. P-Vflowrate_slow curve).
the data processing means are further configured to determine a PEP on the basis of the AOP that has been determined.
the data processing means are further configured to recommend (i.e. determine) a PEP higher than the AOP that has been determined.
the data processing means are further configured to control a display, on the display means, of the recommended PEP.
the AOP value is between 1 and 30 cmH₂O.
the gas supply means comprise a motorized turbine controlled by the data processing means.
the pressure measurement means comprise a pressure sensor and/or the flowrate determination means comprise a flowrate sensor.
it comprises a PEP valve arranged on the patient circuit, in particular on the expiratory branch.
the data processing means are configured to adjust the PEP of said PEP valve to the recommended PEP.
the data processing means are configured to adjust the PEP of said PEP valve in response to activation, by the user, of a setting confirmation means, preferably after the user has pressed a virtual confirmation key displayed on the digital screen.
it comprises electric current supply means for powering the ventilator, such as a connection to the mains (110/220 V) and/or a rechargeable battery.

According to another aspect, the invention also relates to a method for controlling a medical ventilator according to the invention for determining and displaying an AOP value, in particular a medical ventilator as described above, in which method:
A. a determination of the airway opening pressure (AOP) is made by:
   I. supplying the respiratory gas at a “low” respiratory rate of between 2 and 10 ventilation cycles per minute, and at a “slow” insufflation flowrate of between 2 and 10 l/min, until an inspiratory volume of between 200 and 1500 ml or a maximum pressure (Pmax) of between 20 and 60 cmH₂O is obtained,
   II. displaying the stored pressure-volume compliance curve (C1) of the circuit and a pressure-volume curve (C2) corresponding to the slow flowrate insufflation, the slow flowrate insufflation pressure-volume curve (C2) being displayed superimposed on the stored pressure-volume compliance curve (C1) of the circuit,
   III. searching for and locating, on the slow flowrate insufflation pressure-volume curve (C2), an inflection point (PI) corresponding to a change in slope of said slow flowrate insufflation pressure-volume curve (C2) with separation of the slow flowrate insufflation pressure-volume curve (C2) from the pressure-volume compliance curve (C1) of the circuit, and
   IV. determining the pressure value at the inflection point (PI) located on the slow flowrate curve (C1), and
B. the AOP value is displayed that corresponds to the pressure value at the inflection point (PI) determined on the slow flowrate curve (C2).
Depending on the embodiment in question, the method of the invention can comprise one or more of the following features:
   the determination of the AOP is started in response to an actuation of selection means by the user.
   the respiratory gas is supplied at the “low” respiratory rate and at the “slow” insufflation flowrate by controlling the gas supply means.
   the stored pressure-volume compliance curve (C1) of the circuit and the pressure-volume curve (C2) corresponding to the slow flowrate insufflation are displayed on the display means.
   the AOP value is displayed on the display means.
   the medical ventilator comprises gas supply means supplying a patient circuit with respiratory gas, comprising pressure measurement means and flowrate determination means for determining the pressure and flowrate of the gas in the patient circuit; data processing means comprising at least one processor for processing said pressure and/or flowrate measurements; display means; and storage means storing a pressure-volume compliance curve (C1) of the circuit, representing the compliance of the respiratory circuit.
   the respiratory gas is air, oxygen or an air/oxygen mixture.
   the respiratory gas is supplied via gas supply means controlled by the data processing means.
   the gas supply means comprise a motorized turbine.

### BRIEF DESCRIPTION OF THE DRAWINGS

The invention will now be better understood from the following detailed description, which is given by way of non-limiting illustration and with reference to the appended figures, in which:

FIG. 1 shows schematically an embodiment of a medical ventilator according to the invention.

FIG. 2 shows schematically an embodiment of the display of the superimposed pressure-volume curves showing the inflection point that appears in the case of closure of the patient's airways.

### DETAILED DESCRIPTION OF PREFERRED EMBODIMENTS

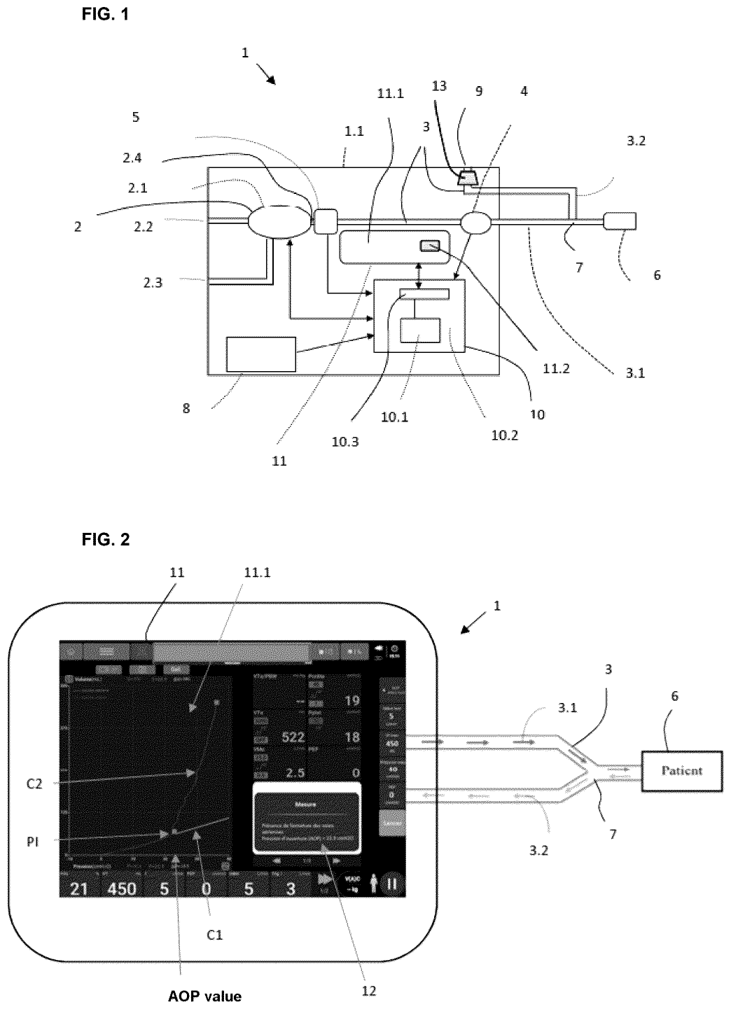

FIG. 1 shows schematically an embodiment of a medical ventilator 1 according to the invention, which comprises gas supply means 2 for supplying a respiratory gas to a patient in need thereof, typically a person suffering from ARDS.

Here, the gas supply means 2 comprise a motorized turbine 2.1 supplied with air via an air delivery conduit 2.2 connected to the ambient atmosphere and with oxygen via an oxygen delivery conduit 2.3 which can be connected to an oxygen source (not shown), such as a pressurized oxygen cylinder or a hospital wall outlet supplied via an oxygen delivery conduit.

However, according to another embodiment (not shown), the gas supply means 2 comprise means for fluidic connection to a respiratory gas source, such as a wall-mounted gas outlet, and one or more controlled valves, such as solenoid valves controlled by the data processing means 10.

Typically, the respiratory gas supplied by the gas supply means 2 is air, oxygen or an air/oxygen gas mixture (>20% vol. O₂, preferably >21% vol. O₂ approximately).

The gas supply means 2 supply a patient circuit 3 comprising an inspiratory branch 3.1 and an expiratory branch 3.2, such as gas passages or conduits, in particular flexible hoses, fluidically connected to a respiratory interface 6, such as a breathing mask or an endotracheal tube, via a joining piece 6, namely a Y-shaped piece or the like.

The inspiratory branch 3.1 delivers the respiratory gas to the respiratory interface 6, which gas is inhaled by the patient during the inspiratory phases, whereas the expiratory branch 3.2 conveys the CO₂-laden gases exhaled by the patient into the respiratory interface 6 during the expiratory phases. The expiratory branch 3.2 is connected to an outlet 9 at which the exhaled gases are discharged from the ventilator 1 and which is fluidically connected to the ambient atmosphere.

A PEP valve 13 is arranged on the patient circuit 3, in particular on the expiratory branch 3.2. The data processing means are configured to adjust the PEP of said PEP valve 13 to the proposed PEP, as explained below, in particular in response to the activation, by the user, of a setting confirmation means, preferably after the user has pressed a virtual confirmation key displayed on the digital screen 11.1.

Furthermore, the patient circuit 3, in particular the inspiratory branch 3.1, comprises pressure measurement means 4, such as a pressure sensor, making it possible to measure, i.e. determine, the pressure of the gas in the inspiratory branch 3.1, and flowrate determination means 5, such as a flowrate sensor, used to determine the flowrate of respiratory gas in the patient circuit 3.

Preferably, the flowrate determination means 5 are arranged close to the outlet 2.4 of the gas supply means 2, that is to say immediately downstream of the turbine 2.1.

Furthermore, the pressure measurement means 4 are situated upstream of the joining piece 6, i.e. the Y-shaped piece.

The medical ventilator 1 comprises data processing means 10, also called control means, comprising one or more microprocessors 10.1 arranged on an electronic board 10.2, which are able to process data and to control elements of the ventilator 1, in particular the operation of the turbine 2.1, the displays on the display screen 11, preferably a touch panel screen 11.1, or others.

The pressure measurement means 4 and the flowrate determination means 5, i.e. the pressure and flowrate sensors, are electrically connected to the data processing means 10, typically to the (micro) processor 10.1, in order to supply them with pressure and flowrate measurements (i.e. values or signals), and the data processing means 10 are configured to process the pressure and flowrate measurements coming from the pressure measurement means 4 and the flowrate determination means 5, i.e. the pressure and flowrate sensors.

The data processing means 10 can, for example, deduce therefrom one or more gas volumes entering or leaving the ventilator 1, typically the patient circuit 3, that is to say respiratory gas supplied to the patient or CO₂-rich gases exhaled by the patient.

The ventilator 1 further comprises storage means 10.3, typically a computer memory, such as a flash memory, RAM or the like, serving to store a pressure-volume compliance curve (i.e. a curve or compliance values making it possible to establish such a curve are stored) of the circuit (P-Vcircuit_patient), representing the compliance of the respiratory circuit (i.e. curve C1 in FIG. 1), or values such as the AOP value. The storage means 10.3 are preferably arranged on the electronic board 10.2 and electrically connected to the (micro) processor 10.1.

Means for supplying electric current, such as a connection to the mains (110/220 V), for example electric cables, and/or a rechargeable battery, power the ventilator 1, in particular the turbine 2.1, the sensors 4, 5, the data processing means 10, the touch screen 11, and the other components that require electric current to function.

All or some of the various components are arranged in an outer casing or shell 1.1 of the ventilator 1.

According to the invention, it is desired to be able to determine a possible closure of the airways in patients, such as ARDS patients, who are ventilated using a medical ventilator 1, such as the one described above, and to measure the airway opening pressure (i.e. the AOP) when present, in order to avoid misinterpretation of the curves and calculations of respiratory mechanics and also to customize the ventilation on the basis of the analysis of the phenomenon of airway closure, and in order to avoid or minimize risks to the patients.

For this purpose, the ventilator 1 is equipped with selection means 11.2 which can be actuated by the user to start a determination of the airway opening pressure or AOP, i.e. to initiate the procedure of determining a possible closure of the patient's airways and the corresponding AOP, as described below.

Preferably, the selection means 11.2 comprise a selection key, in particular a virtual key, i.e. a touch key, displayed on the touch screen 11.1 on which the user presses with a finger, such as the index finger, to initiate the procedure of determining an AOP.

The selection key can be displayed permanently on the screen 11.1 or displayed only after the user, i.e. a caregiver, has chosen an AOP determination procedure from a dedicated menu displayed on the screen 11.1.

According to another embodiment, the ventilator 1 can also comprise a specific button, for example arranged on its casing 1.1, the actuation of which button generates a start of the procedure of determining the opening pressure of the airways, i.e. the AOP.

In all cases, in response to such actuation of the selection means 11.2 by the user, the data processing means 10, typically the processor 10.1, are configured to effect a determination of a possible closure of the patient's airways and, when these are closed, to effect a determination of the corresponding AOP, that is to say initiate a procedure of determination of the AOP.

To do this, the data processing means 10 first control the respiratory gas supply means 2, such as the motorized turbine 2.1, to supply the respiratory gas (i.e. O₂ content>20% vol.), for example air, O₂ or an air/O₂ mixture, at a “low” respiratory rate of between 2 and 10 ventilation cycles per minute, for example of the order of 5 ventilation cycles per minute, and at a “slow” insufflation flowrate of between 2 and 10 l/min, for example of the order of 5 l/min, and this for a duration sufficient to obtain an inspiratory volume of between 200 and 1500 ml or a maximum pressure (Pmax) of between 20 and 60 cmH₂O. The settings for flowrate and inspiratory volume determine the duration. In general, this duration is less than 60 seconds, for example of the order of 1 second to 45 seconds.

Furthermore, the data processing means 10 control a display, on the display means 11, of the pressure-volume compliance curve (curve C1 in FIG. 2) of the circuit (P-Vcircuit_patient) previously stored by the storage means 11.3, and of a pressure-volume curve (curve C2 in FIG. 2) corresponding to the “slow” flowrate insufflation (P-Vflowrate_slow) determined during the given duration.

The “slow” flowrate insufflation pressure-volume curve (P-Vcircuit_patient) (i.e. curve C2), more simply called the “slow flowrate curve”, is displayed superimposed on the stored pressure-volume compliance curve (P-Vcircuit_patient) (i.e. curve C1), more simply called the “compliance curve”, as illustrated in FIG. 2.

As regards the compliance curve (P-Vcircuit_patient), it is determined beforehand, for example during a self-test or similar procedure on the ventilator 1. The compliance of the patient circuit is stored before the start of treatment of the patient, during tests to verify the correct functioning of the machine or “self tests”. This compliance of the circuit is then used to determine and display the compliance curve, i.e. P-Vcircuit_patient curve, superimposed on the slow flowrate curve, i.e. P-Vflowrate_slow curve.

In fact, a self-test procedure on a ventilator 1 is generally performed before ventilating, i.e. treating, a patient, in order to verify the correct functioning of the sensors or other components, and to take account of the characteristics of the patient circuit 3 of the ventilator 1.

To do this, a closure plug can be inserted into the patient circuit 3, typically at the Y-shaped piece 7 of the patient circuit 3, so as to prevent the flow of respiratory gas such as air, delivered by the gas supply means 2, from leaving the circuit 3 via the respiratory interface 6, e.g. mask 6 or the like.

The self-test procedure itself is then launched, for example via a specific selection menu displayed on the screen 11 of the ventilator 1.

This comprises insufflation of a given flowrate of respiratory gas into the inspiratory branch 3.1 of the patient circuit 3, with measurement of the corresponding pressure by the pressure sensor 4, until a set maximum pressure (Pmax) is reached, for example of the order of 60 cmH₂O.

By integrating the gas flowrate, it is then possible to calculate the volume of gas (Vcircuit_patient) delivered in the patient circuit 3.

The data processing means 10, typically the processor 10.1, can then determine the compliance of the circuit (Ccircuit_patient) by using the values of the set maximum pressure (Pmax) and of the delivered volume of gas thus determined (Vcircuit_patient), by carrying out the following calculation: Ccircuit_patient=Vcircuit_patient/Pmax.

In fact, it is considered that compliance is linear, so only two flowrate and pressure measurement points are taken (including the zero flowrate and pressure point). The insufflation flowrate is constant in order to obtain the volume Vcircuit_patient.

Once the circuit compliance has been determined and other internal verification procedures have been effected, the self-test procedure is stopped and the closure plug can be removed so that the ventilator 1 can be used to treat a patient.

The data processing means 10, typically the processor 10.1, are further configured to search for and locate on the slow flowrate curve, i.e. curve C2 in FIG. 2, any inflection point PI from which there is a change in slope of the slow flowrate curve (P-Vflowrate_slow) accompanied by a separation of the slow flowrate curve (i.e. curve C2) from the compliance curve of the circuit (P-Vcircuit_patient) (i.e. curve C1), that is to say a sudden separation of the two curves C1, C2 from each other, as is illustrated in FIG. 2.

If no inflection point PI is found, this means that there is no closure of the patient's airways at this PEP level or that it is not visible at this pressure. Conversely, if an inflection point PI, i.e. inflection point PI in FIG. 2, is found and located on the slow flowrate curve C2, this means that the patient in question has a closure of the airways and the AOP then corresponds to the pressure value at the inflection point PI.

The data processing means 10, typically the processor 10.1, are then configured to determine the pressure value at the inflection point PI that has been located on the “slow” flowrate insufflation pressure-volume curve (P-Vflowrate_slow), i.e. C2 in FIG. 2.

Once determined, the pressure value at the inflection point PI that has been determined is displayed on the display screen 11.1 of the display means 11, given that the AOP corresponds to the pressure value at the inflection point PI of the “slow” flowrate curve (P-Vflowrate_slow), i.e. C2 in FIG. 2.

FIG. 2 shows schematically an example of real-time monitoring of the pressure-volume curves C1, C2 obtained from the pressure and flowrate values processed by the microprocessor 10.1, namely the slow flowrate curve (P-Vflowrate_slow), designated by “C2”, and the compliance curve (P-Vcircuit_patient), designated by “C1”, obtained from the pressure values processed by the microprocessor 10.1 and the compliance of the circuit measured during a self-test procedure, as explained above.

In particular, it is important that the gas is insufflated at a slow flowrate when establishing the slow flowrate curve, since this makes it possible to eliminate the resistive component of the airway pressure, thus making it possible to detect a closure of the patient's airways by locating any abrupt change of slope on the curves (C1, C2), that is to say of the inflection point PI present when the airways are closed but absent when the patient's airways are not closed, which inflection point PI makes it possible to determine the AOP.

In the graph of FIG. 2, the x-axis gives pressure values, expressed in cmH₂O, and the y-axis gives volume values, expressed in ml. Of course, other units/quantities could be used.

It will be seen that an inflection point PI appears where the two curves C1, C2 abruptly deviate from each other, which indicates a closure of the airways of the patient in question and therefore the existence of an AOP.

The AOP value then corresponds to the positive pressure value at the inflection point PI, namely in this case a pressure of 22.9 cmH₂O, as indicated by the x-axis.

This information, namely the existence of an airway closure and the AOP value, is then displayed in a dedicated display window 12 of the display screen 11.1. The display is controlled by the data processing means 10, in particular by the processor 10.1.

The display within the display window 12 can include various items of information useful to the caregiver, in particular:
a text message indicating the presence/existence or absence of an airway closure, for example “presence of airway closure” or “absence of airway closure”; and/or
a text message indicating the value “x” of the AOP in the case of presence of airway closure, for example “AOP=x cmH₂O”, with x between 1 and 30 cmH₂O.

Preferably, the data processing means 10 can also be configured to determine and propose a ventilation strategy, that is to say an action to be taken by the caregiver, as a function of the determined value of the AOP of the patient.

Thus, if the PEP of the PEP valve 13 is set to a value lower than the AOP value, the data processing means 10 can propose a change in the PEP setting to a value higher than the value of the AOP (preferably considered in combination with other available clinical data) and can control a display of this recommended PEP setting on the display means 11.

For example, the data processing means 10 can control the display of a (text) message with a recommendation concerning the PEP setting, for example “maintain PEP” or “increase PEP”, either in the window 12 or elsewhere on the screen 11.1. The recommended PEP value is also advantageously displayed.

By virtue of the invention, with knowledge of the AOP value, the caregiver can adjust the positive expiratory pressure or PEP of the PEP valve 13 to a value above the AOP in order to be able to keep the airways open, so as to limit the risk of lung damage to the patient.

In general, according to the invention, the detection of closure of the patient's airways and the determination of the airway opening pressure (AOP) by analysis of the compliance curve and of the slow flowrate curve (C1, C2), as explained above, are carried out automatically within the medical ventilator 1.

A medical ventilator 1 according to the invention is particularly well suited to the treatment of patients, i.e. men, women or children, for example patients suffering from acute respiratory distress syndrome (ARDS). Indeed, it enables real-time monitoring and detection of the phenomenon of airway closure and determination of the airway opening pressure (AOP) of ventilated patients, such as patients suffering from ARDS.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. Medical ventilator (1) comprising:
gas supply means (2) for supplying a respiratory gas,
a patient circuit (3) supplied with respiratory gas by the gas supply means (2),
pressure measurement means (4) configured to determine the pressure of the gas in the patient circuit (3),
flowrate determination means (5) configured to determine the flowrate of respiratory gas in the patient circuit (3),
data processing means (10) comprising at least one processor (10.1) for processing the pressure and/or flowrate measurements performed by the pressure measurement means (4) and the flowrate determination means (5),
display means (11) controlled by the data processing means (10), and
storage means (10.3) for storing a pressure-volume compliance curve (C1) of the circuit, representing the compliance of the respiratory circuit,
characterized in that:
it further comprises selection means (11.2), which can be actuated by the user in order to start a determination of the airway opening pressure (AOP),
the data processing means (10) are configured to determine the AOP in response to an actuation of the selection means (11.2) by the user:
a) by controlling the gas supply means (2) to supply the respiratory gas at a “low” respiratory rate of between 2 and 10 ventilation cycles per minute, and at a “slow” insufflation flowrate of between 2 and 10 l/min, until an inspiratory volume of between 200 and 1500 ml or a maximum pressure (Pmax) of between 20 and 60 cmH₂O is obtained,
b) by controlling a display, on the display means (11), of the stored pressure-volume compliance curve (C1) of the circuit and a pressure-volume curve (C2) corresponding to the slow flowrate insufflation, the slow flowrate insufflation pressure-volume curve (C2) being displayed superimposed on the stored pressure-volume compliance curve (C1) of the circuit,
c) by searching for and locating, on the slow flowrate insufflation pressure-volume curve (C2), an inflection point (PI) corresponding to a change in slope of said slow flowrate insufflation pressure-volume curve (C2) with separation of the slow flowrate insufflation pressure-volume curve (C2) from the pressure-volume compliance curve (C1) of the circuit, and
d) by determining the pressure value at the inflection point (PI) located on the slow flowrate curve (C1),
and the data processing means (10) are further configured to control a display, on the display means (11), of the AOP value (20) corresponding to the pressure value at the inflection point (PI) that has been determined on the slow flowrate curve (C2).

2. Ventilator according to claim 1, characterized in that the data processing means (10) are further configured to control a display, on the display means (11), of information relating to the existence of a closure of the patient's airways.

3. Ventilator according to claim 1, characterized in that the display means (11) comprise a touch screen (11.1).

4. Ventilator according to claim 1, characterized in that the selection means (11.2) comprise a virtual key displayed on the display means (11), in particular on the touch screen (11.1).

5. Ventilator according to claim 1, characterized in that the data processing means (10) are further configured to control a display, on the display means (11), of a recommended PEP, said recommended PEP being determined on the basis of the AOP (20) that has been determined.

6. Ventilator according to claim 1, characterized in that the AOP value (20) is between 1 and 30 cmH₂O.

7. Ventilator according to claim 1, characterized in that the gas supply means (2) comprise a motorized turbine (2.1) controlled by the data processing means (10).

8. Ventilator according to claim 1, characterized in that the pressure measurement means (4) comprise a pressure sensor.

9. Ventilator according to claim 1, characterized in that the flowrate determination means (5) comprises a flowrate sensor.

10. Ventilator according to claims 1 and 5, characterized in that it comprises a PEP valve (13) arranged on the patient circuit (3), and the data processing means (10) are configured to adjust the PEP of said PEP valve (13) to the recommended PEP.

11. Ventilator according to claim 1, characterized in that the data processing means (10) are configured to control the gas supply means (2) to supply the respiratory gas at a “low” respiratory rate of between 3 and 7 ventilation cycles per minute and/or at a “slow” insufflation flowrate of between 3 and 7 l/min.

12. Ventilator according to claim 5, characterized in that the recommended PEP is higher than the AOP (20) that has been determined.

13. Ventilator according to claim 1, characterized in that the gas supply means (2) are configured to supply a respiratory gas selected from air, oxygen and an air/oxygen mixture.

14. Ventilator according to claim 1, characterized in that the gas supply means supply the respiratory gas at the insufflation flowrate for a duration sufficient to obtain the desired inspiratory volume of between 200 and 1500 ml or the desired maximum pressure (Pmax) of between 20 and 60 cmH₂O.

15. Ventilator according to claim 14, characterized in that said sufficient duration is less than 60 seconds.
